(19) United States
(12) Patent Application Publication
(10) Pub. No.: US 2020/0236986 A1
(43) Pub. Date: Jul. 30, 2020

(54) SMOKE DEVICE COMPRISING CONTAINER

(71) Applicant: I-NVENTION B.V., Delft (NL)

(72) Inventor: Michel Johannes Hendrikus SCHLARMANN, Delft (NL)

(73) Assignee: I-NVENTION B.V., Delft (NL)

(21) Appl. No.: 16/652,573

(22) PCT Filed: Oct. 1, 2018
(86) PCT No.: PCT/NL2018/050647

§ 371 Date: Mar. 31, 2020

(30) Foreign Application Priority Data
Oct. 2, 2017 (NL) 1042565

(51) Int. Cl. A24B 15/167; A24F 1/28; A24D 1/14; A61M 15/06

(52) U.S. Cl. CPC A24B 15/167; A61M 15/06; A24D 1/14; A24F 1/28

(57) ABSTRACT

Device for burning one or more substances contained in a container, such as, for example, tobacco, cannabis or hashish, so that the user can draw in the smoke and vapors released during this process. A heat source such as a gas lighter is used to light the substances to be burned.

The invention relates to a method and device for burning one or more substances present in a container, such as, for example, tobacco, cannabis or hashish, allowing the user to drawn in the smoke and vapors released. A heat source such as a gas lighter is used to light the substances to be burned.

According to the invention, this can be achieved by providing the device with a compartment in which a container can be placed, wherein this container is filled with one or more substances to be burned. The container is provided with perforations on two sides allowing air to be blown through or to be drawn through the container. The device has an air inlet and a vapor outlet, wherein the container is positioned near the entrance.

By positioning a heat source such as, for example, a flame of a lighter, a burning candle or a burning match against, or in the vicinity of, the container, and by sucking at the outlet of the device, the user realizes a heated air flow through the container. As a result, the substances to be burned are heated, optionally until the ignition temperature, enabling the user to draw in the smoke and vapors generated by the device via the outlet of the device.

According to a further aspect of the invention, the device is provided with a container which cannot be filled by the user, but is filled by a supplier with one or more substances to be burned. Accordingly, this container is intended for single use and has been closed by the supplier by means of a press connection, for example.

According to a further aspect of the invention, the device is provided with one or more filters that filter the vapors before these vapours are drawn in. Preferably, the filter is provided with activated carbon, for example.

According to a further aspect of the invention, the device is provided with an additional perforated body in order to prevent ash and/or small parts of the substances to be burned from being drawn in by the user into his mouth.

According to a further aspect of the invention, the device is provided with a container in which a porous body is provided against the wall of the container, wherein the centre of the container is left clear. Advantageously, the porous body is impregnated with a liquid, that will evaporate when hot air, vapour or smoke passes through the centre of the porous body. Preferably, the liquid contains various compositions of terpenes or fragrances or flavors, for example.

Further aspects of the invention that can be used both separately and in combination, are mentioned in the description of the figures and are stated in the claims.

To elucidate the invention, with reference to the drawings, an exemplary embodiment provided with the features is shown and described. Here, the method and the device are shown in a cone shape as an example, but this can also be carried out in one or more different geometries.

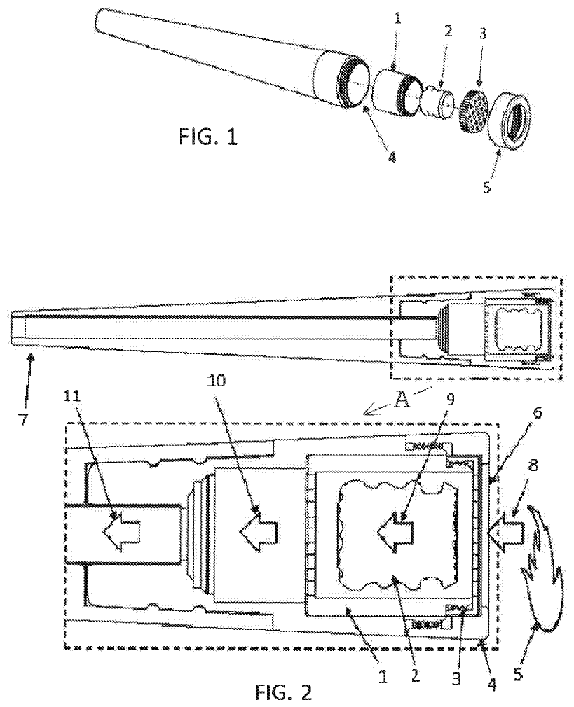

FIG. 1 shows an embodiment of a device according to the invention.

FIG. 1 shows a view of the entire device assembled from a container 1, that is perforated at the bottom, and which is able to receive a substance 2 to be burned, and which is sealed with a perforated cover 3. This closed container construction is positioned in the space 4, after which the entire structure is fixed by the body 5.

FIG. 2 shows a cross-section of the filled and closed device. In detail A, the perforated container 1 is shown, containing the substance 2 to be burned, sealed with the perforated cover 3. A heat source such as a flame 5, for instance of a gas lighter, is positioned near a sealing cap 4, allowing the flame 5 to reach the perforated cover 3 through a recess 6 in the sealing cap 4. By introducing the mouthpiece 7 into his mouth, the user is able to create an underpressure in the entire device, whereby the flame 5 is drawn through the perforated cover 3, which is indicated by means of arrow 8. The heat source 5 will heat the substance 2 to be burned. As a result of the underpressure in the device in the direction of the arrow 9, the vapours released from the substance 2 are drawn in the direction of the arrow 10, at the underside of the perforated container 1, and finally end up in the mouth of the user through the mouthpiece 7 in the direction of the arrow 11, after which the vapour can be either be blown out or drawn in by the user.

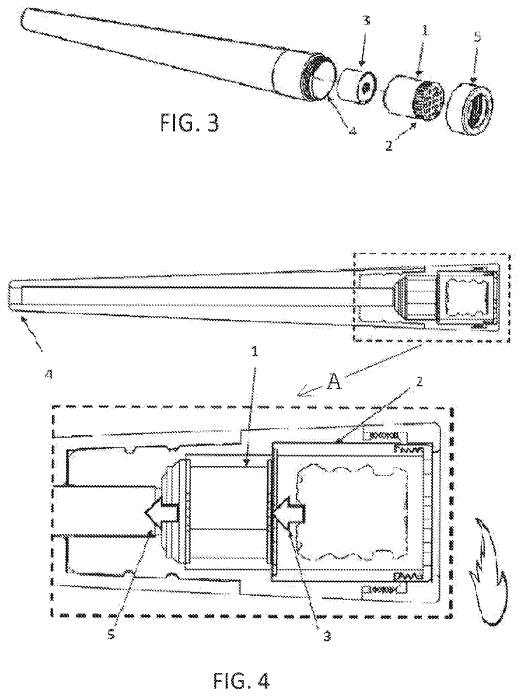

FIG. 3 shows a view of the entire device assembled from a container 1, which is perforated at the bottom. A substance to be burned is received in the container, and the container is sealed with a perforated cover 2. Optionally, a filtering body 3 is positioned in the recess 4., The container 1 with cover 2 is positioned behind the filtering body 3. The entire structure is then fixed by the body 5.

FIG. 4 shows a cross-section of the filled and closed device as shown in FIG. 3. In detail A, a filtering body 1 is shown, which is placed before the container 2. Indicated by arrow 3, vapour and/or smoke, which are extracted from the container 2 by creating an underpressure in the mouthpiece 4, is passed through the filtering body 1 in the direction of arrow 5, through the mouth piece 4, and ends up into the mouth of the user, after which the vapour is either blown out or inhaled by the user.

1. Device for burning one or more substance, such as tobacco, cannabis or hashish, for example, allowing the user to inhale through his mouth the smoke and/or the vapours released,
wherein the device is provided with a compartment (1), which is arranged for receiving a container (2), which container is filled with the one or more substance to be burned.

2. Device according to claim 1, wherein the compartment and/or the container is provided with perforations at least on two sides, thereby allowing air to be blown trough or to be drawn through the compartment and the container.

3. Device according to claim 1, comprising an air inlet and a vapour outlet, wherein the compartment and the container are positioned in the vicinity of the air inlet.

4. Device according to one of the preceding claims, arranged such that a user, by positioning a heat source such as, for example, a flame of a lighter, a burning candle or a burning match against, or in the vicinity of, the container, and by sucking at the outlet of the device, realizes a heated air flow through the container causing heating of the substance to be burned, possibly to the ignition temperature, and enabling the user to draw in the generated smoke and vapours via the outlet of the device.

5. Device according to one of the preceding claims, comprising a container which is not filled by the user, but is filled by a supplier with one or more substance to be burned, which container is intended for single use and has been closed by the supplier by means of a press connection, for example.

6. Device according to one of the preceding claims, provided with one or more filters that filter the vapours before these vapours are drawn in.

7. Device according to claim 5, wherein at least one of said filters is provided with active carbon.

8. Device according to one of the preceding claims, provided with an additional perforated body adapted to prevent ashes and/or small parts of the substance to be burned from being drawn in by the user into its mouth.

9. Device according to one of the preceding claims, provided with a container in which a porous body is provided against the wall of the container, wherein the center of the container is left clear.

10. Device according to claim 9, the porous body being adapted to be impregnated with a liquid that will evaporate when hot air, vapour or smoke passes through the centre of the porous body.

11. Device according to claim 10, wherein the liquid contains various compositions of terpenes or fragrances or flavors, for example.

12. Container (2), adapted to cooperate with a device according to one of the preceding claims, which container comprises a closed housing of porous material and/or of material that is permeable for air or vapour and/or smoke, and which container is filled with one or more substance to be burned in the device during the stay of the container in said compartment (1) of the device arranged for receiving the container.
